(19) United States
(12) Patent Application Publication
(10) Pub. No.: US 2023/0082714 A1
(43) Pub. Date: Mar. 16, 2023

(54) Soil Property Model Using Measurements of Properties of Nearby Zones

(71) Applicant: FARMERS EDGE INC., Winnipeg (CA)

(72) Inventors: David Eric Chalmers, Winnipeg (CA); Fatema Tuz Zohra, Winnpeg (CA); Guy Duke, Winnipeg (CA); Kevin Grant, Winnipeg (CA)

(21) Appl. No.: 17/939,126

(22) Filed: Sep. 7, 2022

Related U.S. Application Data
(60) Provisional application No. 63/243,263, filed on Sep. 13, 2021.

(51) Int. Cl. G01N 33/24

(52) U.S. Cl. CPC G01N 33/24; G01N 2033/245

(57) ABSTRACT

In an agricultural field having first regions with current soil test values known from actual tests and second regions having unknown soil test values, nutrient levels are predicted using a soil test model which defines a statistical relationship between (i) nutrient levels for a given region of a training field for a given season and (ii) field specific characteristics for the given region in a previous growing season and nutrient levels in the given region or proximate regions from the given growing season. Acquired known field specific characteristics and current soil test values from the first regions are then applied to the soil test model to calculate the predicted nutrient level the second regions. This can reduce the cost of soil sampling by using actual soil test results from one management zone as a predictor when modeling other zones' properties.

This application claims the benefit under 35 U.S.C. 119(e) of U.S. provisional application Ser. No. 63/243,263, filed Sep. 13, 2021.

### FIELD OF THE INVENTION

The present invention relates to a method of predicting soil properties, for example nutrient levels, in unknown regions or zones of an agricultural field using a model that uses current soil test data of nearby regions or zones within the same agricultural field as an input.

### BACKGROUND

Precision agronomists often attempt to divide a field or agricultural area into “management zones”—geographic divisions which are predicted to have roughly homogeneous soil characteristics and yield potential. Taking soil samples from each zone then allows fertilizer prescriptions to be tailored to that zone, optimizing production and economic return for the farmer. Taking soil samples is labor intensive and can be both costly and time consuming when required for an agricultural field having many large sized zones.

### SUMMARY OF THE INVENTION

According to one aspect of the invention there is provided a method of predicting soil nutrient levels for a current growing season in a common agricultural field having a plurality of regions including at least one first region having a current soil test value that is known from an actual soil test and at least one second region having a current soil test value that is unknown, the method comprising:
receiving a request for a nutrient level in said at least one second region;
providing a soil test model which defines a statistical relationship between:
   (i) nutrient levels for a given region of a training agricultural field in a given growing season, and
   (ii) field specific characteristics for the given region in a previous growing season prior to the given growing season and nutrient levels in one or more of the given region or a proximate region of the training agricultural field from the given growing season;
acquiring known field specific characteristics from a prior growing season prior to the current growing season for said at least one second region;
acquiring the current soil test value for said at least one first region;
applying said known field specific characteristics and said current soil test value from said at least one first region to the soil test model to calculate a predicted nutrient level for said at least one second region; and
transmitting the predicted nutrient level to a user.

According to a second aspect of the present invention there is provided a system comprising one or more processors and one or more memories storing computer program instructions for predicting soil nutrient levels for a current growing season in a common agricultural field having a plurality of regions including at least one first region having a current soil test value that is known from an actual soil test and at least one second region having a current soil test value that is unknown, the system, when executing the computer program instructions by the one or more processors, being configured to:
receive a request for a nutrient level in said at least one second region;
provide a soil test model which defines a statistical relationship between:
   (i) nutrient levels for a given region of a training agricultural field in a given growing season, and
   (ii) field specific characteristics for the given region in a previous growing season prior to the given growing season and nutrient levels in one or more of the given region or a proximate region of the training agricultural field from the given growing season;
acquire known field specific characteristics from a prior growing season prior to the current growing season for said at least one second region;
acquire the current soil test value for said at least one first region;
apply said known field specific characteristics and said current soil test value from said at least one first region to the soil test model to calculate a predicted nutrient level for said at least one second region; and
transmit the predicted nutrient level to a user.

It is possible to reduce the cost of soil sampling by instead modeling the soil properties in each zone, using e.g., a process-based or machine-learning-based modeling approach that considers previous seasons' soil sample properties and the weather, fertilizer, cropping and yield history of that zone. Here we propose a method of improving the soil-property modeling accuracy, by using actual soil test results from one management zone as a predictor when modeling other zones' properties.

The system and/or method may further include training the soil test model using a machine learning algorithm with soil test values and field specific characteristics associated with a plurality of different training agricultural fields at different stages throughout one or more growing seasons.

The system and/or method may further include training the soil test model using data from a training agricultural field having a plurality of regions including at least one known region in which a soil test value is known from an actual soil test and at least one unknown region in which the soil test value is unknown, by assigning a virtual value as the soil test value for said at least one unknown region based upon the soil test value of said at least one known region.

The step of assigning the virtual value as the soil test value for said at least one unknown in the system and/or method may further comprise (i) ranking the known regions according to productivity, and (ii) using the soil test value of a median region among the ranked known regions as the virtual test value assigned to the soil test value for said at least one unknown region.

The step of assigning the virtual value as the soil test value for said at least one unknown in the system and/or method may comprise (i) ranking the known regions according to productivity and (ii) using the soil test value of a median region among the ranked known regions as one of the predictors for estimating the soil test value for the unknown regions.

The step of training the soil test model in the system and method may further comprise assigning the soil test value of the median region as the soil test value for each known region. In this instance, the step of training the soil test model may further comprise assigning the soil test value from one of the known regions adjacent to the median region as the soil test value for the median region.

The system or method may be further arranged such that for each known region having data from a plurality of soil tests associated therewith, an average value is calculated from said data and the average value is assigned as the soil test value for that known region.

The known field specific characteristics may include: (i) the nutrient levels acquired from soil tests performed during the prior growing season, (ii) weather data relating to common agricultural field during either or both of the current growing season and the prior growing season, (iii) soil characteristics other than nutrient levels, measured in-field during either or both of the current growing season and the prior growing season, (iv) remotely sensed data acquired during either or both of the current growing season and the prior growing season, (v) harvest layer information associated with either or both of the current growing season and the prior growing season, (vi) yield values associated with either or both of the current growing season and the prior growing season, (vii) agronomist recommendations associated with either or both of the current growing season and the prior growing season, (viii) fertilizer applications associated with either or both of the current growing season and the prior growing season, and/or (ix) any combination of the above characteristics.

The step of providing the soil test model in the system and/or method may further comprise (i) selecting one or more field specific characteristics among a plurality of field characteristics available for the training agricultural field using an embedded feature selection approach, and (ii) training the soil test model to define said statistical relationship using the selected field specific characteristics.

The system or method may be further arranged such that for each first region of the common agricultural field having data from a plurality of actual soil tests associated therewith, an average value is calculated from said data and the average value is assigned as the current soil test value for that first region.

### BRIEF DESCRIPTION OF THE DRAWINGS

The Figures (FIGS.) and the following description relate to preferred embodiments by way of illustration only. It should be noted that from the following discussion, alternative embodiments of the structures and methods disclosed herein will be readily recognized as viable alternatives that may be employed without departing from the disclosed principles. It is noted that wherever practicable similar or like reference numbers may be used in the figures and may indicate similar or like functionality. The figures depict embodiments of the disclosed system (or method) for purposes of illustration only.

One embodiment of the invention will now be described in conjunction with the accompanying drawings in which:

FIG. 1 illustrates a system environment for running Virtual Soil Test (VST) processes over an agricultural field using season soil test results, weather, cropping, harvest, applications, etc., according to one example embodiment.

FIG. 2 illustrates the difference between the standard VST approach and the VST model which incorporates current soil test values.

FIG. 3 illustrates the processes required to run VST Module according to the present invention.

In the drawings like characters of reference indicate corresponding parts in the different figures.

### DETAILED DESCRIPTION

According to the present invention, a standard Virtual Soil Testing (VST) model uses machine learning techniques to derive statistical relationships between a zone's previous-season agronomic details (previous-season soil test results, weather, cropping, harvest, applications, etc.) and the current season's soil properties. The improved model leverages the zoning process that we already use; realizing that soil properties measured in one zone can be helpful in predicting properties for other zones in the same field. The new model considers the same predictors as the standard model, but additionally considers the current-season soil properties measured in other zones in the same field and could also consider properties of that zone relative to the one being predicted for. The new relationships learned by the machine learning process includes information about the relationships between zones' properties, resulting in more accurate predictions whenever a soil sample from a nearby zone is available. Operationally, this means we can scale back from sampling every zone in a field, to just sampling one and using the result to enhance the predictions in the other zones.

An overview of the processes includes:

Training

(i) Take all zones in a management zone with actual soil tests (ASTs).

(ii) Select the median zone number in which zone numbers are in order of increasing quality. So, this is selecting the median quality zone.

(iii) Also select the median zone number+1.

(iv) Create the infield AST feature by: (a) For every zone except the median zone, the infield value is the AST from the median zone; and (b) For the median zone, the infield value is the median zone number+1.

Prediction

(i) Expect that each management zone has at least one AST.

(ii) Use the average of the ASTs as the infield AST feature.

VST System Environment 100

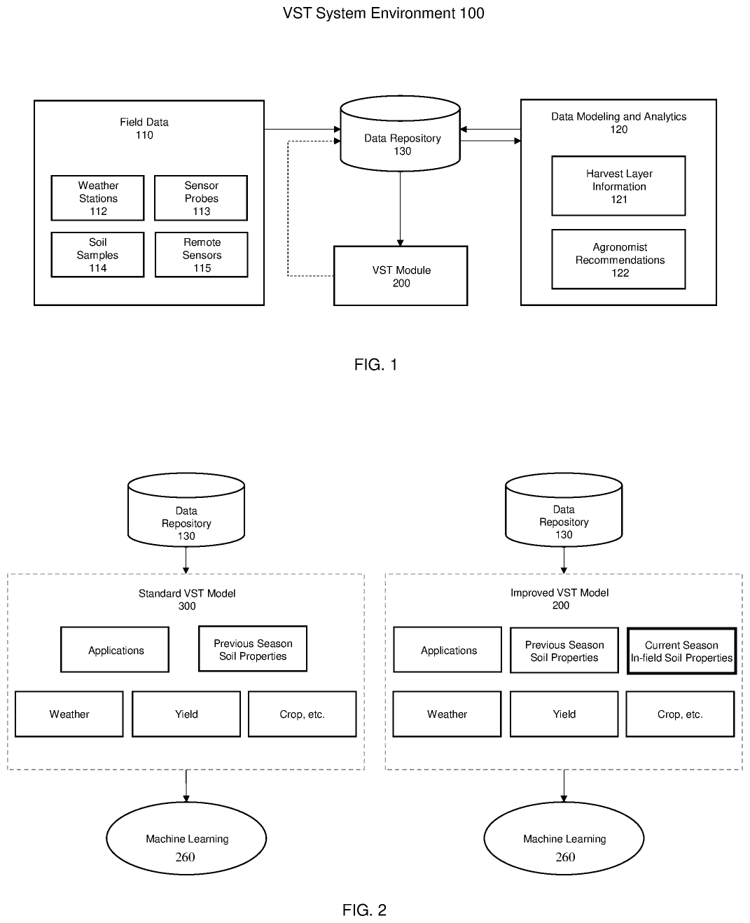

FIG. 1 illustrates a system environment for running Virtual Soil Test (VST) processes over an agricultural field using season soil test results, weather, cropping, harvest, applications, etc., according to one example embodiment. The VST system environment 100 includes data from different sources such as field data 110, harvest layer information 121 and recommendation engine 122, the data repository 130, and the VST module 200. Other examples of a system environment are possible. For example, in various embodiments, the system environment 100 may include additional or fewer systems.

Field data 110 can be data acquired from (a) weather stations 111 (data can include, for example, precipitation, daily and hourly precipitation, temperature, wind gust, wind speed, pressure, clouds, dew point, delta T, GFDI, relative humidity, historical weather, forecast, wind direction, barometric pressure, growing degree days, humidity), (b) sensor probes 112 (for example, a soil moisture probe that provides near-real-time data on volumetric soil moisture content, which gets converted into percent available water within the crop rooting zone, inches of available water, crop root dynamics, and irrigation requirements; the probe also measures soil temperature at various depths), (c) soil samples 113 (data can include, for example, elemental composition, pH, organic matter, cation exchange capacity, percent base saturation, excess lime, soluble salts), and (d) remote sensors 114 (for example, sensors on farm structures, drones, and robots).

Data collected in the data repository 130 is processed to derive value from data that can drive functions such as visualization, reports, decision making, and other analytics. Functions created may be shared and/or distributed to authorized users and subscribers. The processing of data occurs in data modeling and analytics 120. Some authorized users or devices may be given authorization to only view the data stored in the data repository 130, not change it. Other authorized users or devices may be given authorization to both view/receive data from and transmit data into the data repository 130.

Data modeling and analytics 120 may be programmed or configured to manage read operations and write operations involving the data repository 130 and other functional elements of a precision agricultural system. For example, in FIG. 1, the data modeling and analytics 120 includes harvest layer information 121, agronomist recommendations 122 which provides processed data and agronomics analytics that can be stored in the data repository 130 to be used by authorized users or devices.

The VST module 200 collects all these data from data repository 130 to run the VST process. If certain criteria are met, the VST module will generate predictions for the requested fields using the trained model so that the results can be transmitted to the authorized user.

VST Module 200

A goal of precision farming is to improve site-specific agricultural decision making through collection and analysis of data, formulation of site-specific management recommendations, and implementation of management practices to correct the factors that limit crop growth, productivity, and quality (Mulla and Schepers, 1997). Management zones are used in precision farming to divide a field or agricultural area into geographic divisions which are predicted to have homogenous soil properties and fertility levels. The process of “zoning” a field presents an opportunity to physically sample one zone and use the results to make better virtual samples of the other zones. This gives a nice balance between cost savings (reducing the number of physical samples) and accuracy of the virtual samples. Methods and systems in this disclosure improve the soil-property modeling accuracy, by using actual soil test results from one zone along with field-specific data, previous season soil test results, recommended fertilizer, crop and yield history, etc. as predictors when modeling other zones' soil properties.

The difference between the standard VST approach and the new VST model is depicted in FIG. 2. The standard VST model interpret the relationships between a zone's previous-season agronomic details (previous-season soil test results, weather, cropping, harvest, applications, etc.) and the current season's soil properties. The new model considers the current-season soil properties measured in other zones in the same field along with the other predictors used in the standard model. The new relationships learned by the machine learning process results in more accurate predictions whenever a soil sample from a nearby zone is available.

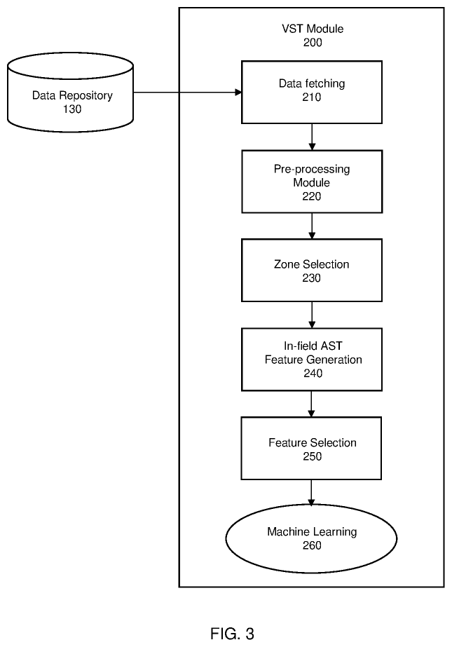

FIG. 3 illustrates the processes required to run VST Module 200. The VST Module uses machine learning techniques to derive relationships between a zone's previous-season agronomic details and the current-season soil properties. VST Module can utilize any field-specific data, previous-season soil test results, agronomist recommendations, etc. to predict the current-season soil properties. The VST model using the current-season soil properties from a different zone in the same field results in improved predictions.

Data Fetching 210

Data fetching module 210 collects various field-specific features and weather data from the data repository 130. Data from various sources such as weather stations 111, sensor probes 112, remote sensors 114, etc. are stored in data repository 130. For generating features for the VST method, field-specific properties such as fertilizer, cropping and yield history, soil sample properties, the weather data and previous-season agronomic details are collected from the data repository 130 by the data fetching module 210.

Examples of data fetched for the VST methods can be as follows: (i) Weather data (including daily and hourly precipitation, temperature, wind gust, wind speed, wind pressure, wind direction, cloud percentage, dew point, relative humidity, barometric pressure, solar radiation, and relative humidity); (ii) Crop information (variety, previous crops, seeding date, etc.); (iii) Regional soil characteristics; (iv) Previous and current season Soil test results; (v) Agronomist recommendation (for example, applied fertilizer amount); and (vi) Yield and harvest history.

Preprocessing Module 220

In the pre-processing module 220, the data collected in the data fetching module 210 is processed to make them ready for further processing. A series of filters are applied to the dataset to remove the anomalous data. There are few preprocessing steps such as null value imputation, one-hot encoding, dropping highly correlated features, feature engineering, etc. are applied to the input data in this module.

Zone Selection 230

For an in-field VST method, a zone needs to be selected to use the feature from that zone to generate the in-field feature for the other zones within a management zone. The zone numbers within a management zone are in order of increasing order. All the zone numbers within a management zone with actual soil test (AST) results are collected to select the median zone number and the median zone number+1 to use the properties from those zones as the AST feature for the other zones.

In-Field AST Feature Generation 240

In-field AST feature generation is the process of generating features from the current season soil properties from a zone to generate features for other zones in the same field. For every zone except the median zone, the infield value is the AST from the median zone selected from the zone selection module 230. And, for the median zone, the infield value is the median zone number+1. For example, for subfields with two zones, the infield AST feature values are the swapped values of the ASTs and for subfields with three zones, the infield AST feature for zones 1 and 3 is the value of zone 2's AST. The infield AST feature for zone 2 is the value of zone 3's AST.

Feature Selection 250

For selecting the best features for the VST method, a feature selection approach is adopted. Before feeding the features to a machine learning model, the important features are selected by using the feature selection module 250. There are various feature selection algorithms available in the literature such as filter method, wrapper method, embedded method, etc. In one example embodiment of the VST method, embedded feature selection approach is used to select the important features for the VST model.

Machine Learning Module 260

Machine learning module 260 is a process-based or machine-learning-based modeling approach that considers previous seasons' soil sample properties and the weather, fertilizer, cropping and yield history of that zone. The in-field AST is an improvement over the standard VST approach by using actual soil test results from one management zone as a predictor when modeling other zones' properties. All these features are fed to a machine learning model to interpret the relationships between a zone's previous-season agronomic details and the current-season soil properties. A few examples of commonly used algorithms that can be used in this machine learning module 260 for interpreting the relationship are eXtreme Gradient Boosting (XGBoost), Neural Network or any tree-based algorithm.

Model Training

For training the VST model, the selected features are fed to the machine learning model. A 5-fold cross-validation is performed to select the best-fitted model. The accuracy of the models is evaluated using the mean absolute error (MAE).

Prediction

When predicting the soil properties for a field or management zone, all the field-specific features, weather data, crop and yield history are pulled from the data repository 130. For generating the in-field AST feature, it is expected that each management zone will have at least one actual soil test result. In the case of more than one AST, the average of all the ASTs is considered as the infield AST feature. The features selected in the model training are taken from the list of all the features and fed to the trained model to generate the prediction of the soil properties for the current season.

Once the prediction of the soil properties has been determined, it may be accessed by the grower or authorized third-party entities. Furthermore, this information may be sent to the grower or authorized third-party entities in the form of a notification.

Since various modifications can be made in this invention as herein above described, and many apparently widely different embodiments of same made, it is intended that all matter contained in the accompanying specification shall be interpreted as illustrative only and not in a limiting sense.

1. A method of predicting soil nutrient levels for a current growing season in a common agricultural field having a plurality of regions including at least one first region having a current soil test value that is known from an actual soil test and at least one second region having a current soil test value that is unknown, the method comprising:
receiving a request for a nutrient level in said at least one second region;
providing a soil test model which defines a statistical relationship between:
(i) nutrient levels for a given region of a training agricultural field in a given growing season, and
(ii) field specific characteristics for the given region in a previous growing season prior to the given growing season and nutrient levels in one or more of the given region or a proximate region of the training agricultural field from the given growing season;
acquiring known field specific characteristics from a prior growing season prior to the current growing season for said at least one second region;
acquiring the current soil test value for said at least one first region;
applying said known field specific characteristics and said current soil test value from said at least one first region to the soil test model to calculate a predicted nutrient level for said at least one second region; and
transmitting the predicted nutrient level to a user.

2. The method according to claim 1 including training the soil test model using a machine learning algorithm with soil test values and field specific characteristics associated with a plurality of different training agricultural fields at different stages throughout one or more growing seasons.

3. The method according to claim 1 including training the soil test model using data from a training agricultural field having a plurality of regions including at least one known region in which a soil test value is known from an actual soil test and at least one unknown region in which the soil test value is unknown, by assigning a virtual value as the soil test value for said at least one unknown region based upon the soil test value of said at least one known region.

4. The method according to claim 3 wherein the step of assigning the virtual value as the soil test value for said at least one unknown further comprises:
ranking the known regions according to productivity;
using the soil test value of a median region among the ranked known regions as the virtual test value assigned to the soil test value for said at least one unknown region.

5. The method according to claim 3 wherein the step of assigning the virtual value as the soil test value for said at least one unknown further comprises:
ranking the known regions according to productivity;
using the soil test value of a median region among the ranked known regions as one of the predictors for estimating the soil test value for the unknown regions.

6. The method according to claim 3 wherein the step of training the soil test model further comprises:
assigning the soil test value of the median region as the soil test value for each known region.

7. The method according to claim 6 wherein the step of training the soil test model further comprises:
assigning the soil test value from one of the known regions adjacent to the median region as the soil test value for the median region.

8. The method according to claim 3 further comprising:
for each known region having data from a plurality of soil tests associated therewith, calculating an average value from said data and assigning the average value as the soil test value for that known region.

9. The method according to claim 1 wherein said known field specific characteristics include the nutrient levels acquired from soil tests performed during the prior growing season.

10. The method according to claim 1 wherein said known field specific characteristics include weather data relating to common agricultural field during either or both of the current growing season and the prior growing season.

11. The method according to claim 1 wherein said known field specific characteristics include soil characteristics other than nutrient levels, measured in-field during either or both of the current growing season and the prior growing season.

12. The method according to claim 1 wherein said known field specific characteristics include remotely sensed data acquired during either or both of the current growing season and the prior growing season.

13. The method according to claim 1 wherein said known field specific characteristics include harvest layer information associated with either or both of the current growing season and the prior growing season.

14. The method according to claim 1 wherein said known field specific characteristics include yield values associated with either or both of the current growing season and the prior growing season.

15. The method according to claim 1 wherein said known field specific characteristics include agronomist recommendations associated with either or both of the current growing season and the prior growing season.

16. The method according to claim 1 wherein said known field specific characteristics include fertilizer applications associated with either or both of the current growing season and the prior growing season.

17. The method according to claim 1 wherein the step of providing the soil test model further comprises:
selecting one or more field specific characteristics among a plurality of field characteristics available for the training agricultural field using an embedded feature selection approach; and
training the soil test model to define said statistical relationship using the selected field specific characteristics.

18. The method according to claim 1 further comprising:
for each first region of the common agricultural field having data from a plurality of actual soil tests associated therewith, calculating an average value from said data and assigning the average value as the current soil test value for that first region.

19. A system comprising one or more processors and one or more memories storing computer program instructions for predicting soil nutrient levels for a current growing season in a common agricultural field having a plurality of regions including at least one first region having a current soil test value that is known from an actual soil test and at least one second region having a current soil test value that is unknown, the system, when executing the computer program instructions by the one or more processors, being configured to:
receive a request for a nutrient level in said at least one second region;
provide a soil test model which defines a statistical relationship between:
(i) nutrient levels for a given region of a training agricultural field in a given growing season, and
(ii) field specific characteristics for the given region in a previous growing season prior to the given growing season and nutrient levels in one or more of the given region or a proximate region of the training agricultural field from the given growing season;
acquire known field specific characteristics from a prior growing season prior to the current growing season for said at least one second region;
acquire the current soil test value for said at least one first region;
apply said known field specific characteristics and said current soil test value from said at least one first region to the soil test model to calculate a predicted nutrient level for said at least one second region; and
transmit the predicted nutrient level to a user.
